(19) United States
(12) Patent Application Publication
(10) Pub. No.: US 2009/0082837 A1
(43) Pub. Date: Mar. 26, 2009

(54) Hand-held Thermal Ablation Device

(76) Inventors: Barry N. Gellman, N. Easton, MA (US); Jozef Slanda, Milford, MA (US); Armand Morin, Berkley, MA (US)

Correspondence Address: FAY KAPLUN & MARCIN, LLP, 150 BROADWAY, SUITE 702, NEW YORK, NY, 10038, US

(21) Appl. No.: 12/209,460

(22) Filed: Sep. 12, 2008

Related U.S. Application Data
(60) Provisional application No. 60/973,907, filed on Sep. 20, 2007.

(51) Int. Cl. A61F 7/12; A61B 17/42

(52) U.S. Cl. 607/105; 606/119; 604/43

(57) ABSTRACT

A thermal ablation system, comprises a device housing and an elongated probe extending distally from the device housing, the probe including an outflow fluid passage extending between proximal and distal outflow openings and a return fluid passage extending between proximal and distal return openings, the probe being shaped and sized for insertion into a body lumen so that, when the distal outflow and return openings are located at a desired position within the body, the proximal outflow and return openings remain outside the body in combination with a pump disposed in the device housing in fluid communication with the outflow and return fluid passages of the probe for circulating a fluid through the outflow lumen into a target area of the body and back through the return lumen to the device housing and a heating element in the device housing for heating the fluid. Fluid connectors place the pump and the heating element in fluid communication with a supply of fluid and a fluid drain.

### PRIORITY CLAIM

This application claims the priority to the U.S. Provisional Application Ser. No. 60/973,907, entitled “Hand-Held Thermal Ablation Device,” filed Sep. 20, 2007. The specification of the above-identified application is incorporated herewith by reference.

### BACKGROUND

Conventional treatments for uterine fibroids include drug therapies that are generally better suited for less advanced cases and hysterectomies for more advanced cases. However, less invasive alternative procedures are often preferable to the hysterectomy as they typically reduce side effects, hospital stays, and discomfort.

These less invasive procedures have employed electrical energy (e.g., RF energy), heat and cryogenic treatments, as well occlusion of the blood supply to the fibroids. Alternatively, the entire inner lining of the uterus may be treated by, for example, conduction uterine ablation—i.e., circulating a heated fluid within the uterus.

The Hydro-Therm Ablator (HTA)™ system marketed by the Boston Scientific Corporation ablates the uterine lining by circulating saline heated to between approximately 41.5° and 99.9° C. for about 10 minutes. The system incorporates a hand-held probe for insertion into the uterus connected by tubing extending to an external device containing heating elements and a pump. In other similar systems, the heated fluid may be contained within a balloon while circulating within the uterus.

### SUMMARY OF THE INVENTION

In one aspect, the present invention is directed to a thermal ablation system, comprising an elongated probe extending distally from a device housing, the probe including an outflow fluid passage extending between proximal and distal outflow openings and a return fluid passage extending between proximal and distal return openings, the probe being shaped and sized for insertion into a body lumen so that, when the distal outflow and return openings are located at a desired position within the body, the proximal outflow and return openings remain outside the body and a pump disposed in the device housing in fluid communication with the outflow and return fluid passages of the probe for circulating a fluid through the outflow lumen into a target area of the body and back through the return lumen to the device housing in combination with a heating element in the device housing for heating the fluid and fluid connectors placing the pump and the heating element in fluid communication with a supply of fluid and a fluid drain.

In another aspect, the present invention is directed to a hand-held thermal ablation device, comprising an elongated probe extending between a proximal end coupled to a handle and a distal end which, when in an operative position, is received within a body lumen and a pump within the handle in fluid connection with fluid passages in the elongated probe in combination with a fluid column within the handle containing a heating element, serially connected with the pump and an external fluid supply in fluid connection with the pump.

In a still further aspect, the present invention is directed to a method of ablating target tissue, comprising advancing a distal end of an elongated probe of a hand-held device into a body lumen and heating a fluid with a heating element disposed in a housing of the hand-held device in combination with the steps of motivating the fluid with a pump disposed in the housing, to inject the fluid into the body lumen via an outflow passage of the elongated probe to ablate target tissue therein and withdrawing the fluid from the body lumen via a return passage of the elongated probe.

### BRIEF DESCRIPTION OF DRAWINGS

FIG. 1 is a drawing of an embodiment of a hand-held thermal ablation apparatus according to the invention;

FIG. 2 is a detail view of a handle of the thermal ablation apparatus shown in FIG. 1;

FIG. 3 is a drawing of a second embodiment of a hand-held thermal ablation system according to the invention;

FIG. 4 is a diagram showing fluid flow through the thermal ablation system of FIG. 3;

FIG. 5 is an exploded view showing a reservoir and impeller of the embodiment of FIG. 3;

FIG. 6 is an exploded view showing a reservoir assembly and a motor housing of the embodiment of FIG. 3;

FIG. 7 is a photograph of a detail of the fluid reservoir and electrode of the embodiment of FIG. 3;

FIG. 8 is a detail view of a cap with a heating element of the embodiment shown in FIG. 3.

FIG. 9 is a diagram showing the reservoir, upper pump and outlet port of the embodiment shown in FIG. 3;

FIG. 10 is a photograph of the impeller of the thermal ablation system shown in FIG. 3;

FIG. 11 is a detail view showing the handle inlet and outlet of the embodiment shown in FIG. 3;

FIG. 12 is a photograph of a further embodiment of a heating and pump unit of a hand-held thermal ablation apparatus according to the invention; and

FIG. 13 is a cutaway diagram showing an integrated hand-held thermal ablation device according to the invention.

### DETAILED DESCRIPTION

The present invention may be further understood with reference to the following description and to the appended drawings, wherein like elements are referred to with the same reference numerals. The present invention relates to devices for treating fibroids or other target tissue in a hollow organ. In particular, the present invention relates to devices for ablating the lining of the uterus.

The embodiments of the present invention provide a compact, hand-held device for ablation of the lining of a hollow organ, such as the uterus. The system according to the invention comprises a hand-held probe connected to a fluid supply with a pump and heater contained within the hand-held housing for motivating and heating the fluid as necessary.

The exemplary uterine probe is inserted through the vaginal canal and the cervix to place a distal tip thereof within the uterus. During the therapy, the distal tip of the probe which contains inflow and outflow orifices is located within the uterus just distal to the internal cervical os. In one embodiment, the probe uses a coaxial design for the inflow and outflow passages. The fluid passages are connected to a pump that provides aspiration of return fluid and which imparts energy to force fluid through the probe out of the outflow orifice and into the uterus.

The exemplary device also comprises a fluid channel or reservoir and a fluid heater, in line with the pump. In this embodiment, the heater is selected to provide a supply of fluid heated to, for example, approximately 90° C. for about 10 minutes. However, those skilled in the art will understand that other temperatures and/or durations may be selected to adapt the system to the requirements of particular procedures through simple adjustment and/or replacement of components such as heating elements and power supplies. For example, ablation may be carried out using any fluid temperature between approximately 41.5° and 99.9° C. with the time required to achieve a desired degree of ablation increasing as the fluid temperature decreases. As would be understood by those skilled in the art, as tissue is ablated, it becomes an insulator so that the duration of heat must be increased necrose deeply (e.g., to shut down blood flow through vessels supplying fibroids). According to the invention, the exemplary fluid circuit may also comprise a temperature probe to monitor fluid temperature and a feed back loop to shut down the pump and/or the heater when the fluid temperature exceeds a preset level. The feed back loop is an important safety feature designed to prevent injury from excessive heating of the fluid.

A fluid flow sensor and/or indicator may also be included in the exemplary flow path, and may be associated to a sensor feedback loop to the pump motor to control the outflow of the pump (e.g., when an amount of fluid input to the uterus exceeds an amount withdrawn by more than a predetermined level). The pump may also be manually controlled via an on/off switch and a circulation adjustment controller for user controlled fluid circulation. As will be described in greater detail below, the flow passages may comprise a fluid fill port, a fluid drain port, an air vent port and a compliance chamber to enable reserve fluid to maintain the uterus full of fluid. The compliance chamber may be located within the handle or between the handle and the fluid feed from the priming bag.

Saline may be advantageously circulated through the system according to the invention. However, it will be understood by those of skill in the art that a variety of other fluids such as glycerine, may be used without departing from the teachings of the invention. Preferably, the fluid is osmotically safe so that it will not change the electrolyte balance of the blood as it is absorbed into tissue over time. If an RF electrode is to be included in the heating element, an electrically conductive fluid (e.g., saline) is preferably selected. In addition to an RF heating element or as an alternative thereto, a cartridge and/or resistance heater may be used to bring the circulating fluid to the desired temperature even for fluids such as glycerine which are not electrically conductive.

A more detailed description of exemplary embodiments of the invention is presented below. The exemplary device utilizes coaxial conduits to circulate fluid into and out of the uterus providing single point access to the uterus via the cervix. The device is manually positioned and held in place by the user during the length of the therapy, typically about 10 minutes. The hand-held probe passes through the cervix with outer surfaces of an insertion section of the probe sealing the internal cervical os as well as the external cervical os. Inlet and outlet fluid ports are located at the distal end of the probe to circulate the heated fluid into the uterus and to withdraw fluid therefrom.

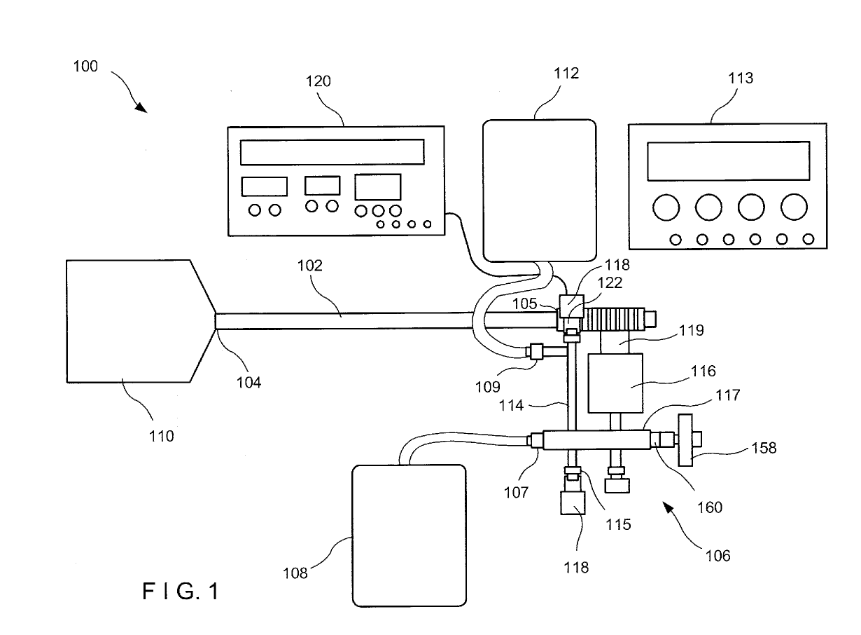

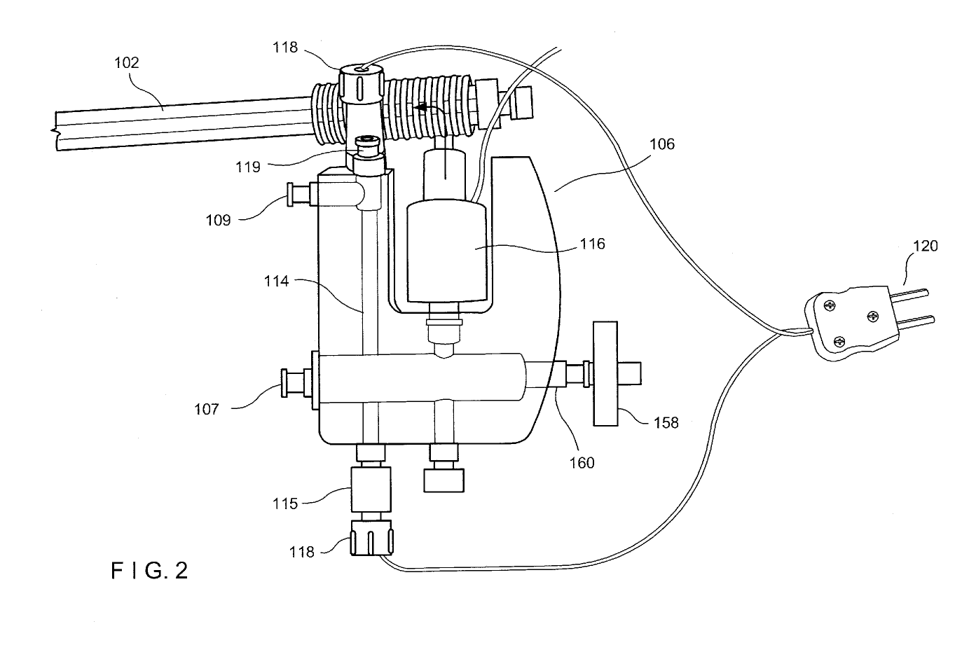

FIGS. 1 and 2 show an exemplary embodiment of a hand-held thermal ablation (HTA) system 100 including an elongated probe body 102 extending from a distal tip 104 to a proximal end 105 which is coupled to a handle 106. The handle 106 which extends substantially perpendicular to an axis of the probe body 102 includes an inlet port 107 for coupling to a source of ablation fluid such as an IV bag 108 and an outlet port 109 for coupling to a drainage reservoir such as a drainage bag 112. The handle 106 also includes a pump 116 (e.g., an oscillating pump) which circulates fluid from the bag 108, through the probe body 102 into the uterus and back through the probe body 102 to the drainage bag 112 via the outlet port 109. As would be understood by those skilled in the art, the pump 116 may be powered by a DC power supply 113 connected thereto by conventional means or, alternatively, by a battery or other power source contained within the handle 106.

The handle 106 also includes a heating column 114 including a pair of electrodes 118 which receive power from an RF generator 120 to heat fluid passing through the heating column 114. As would be understood by those skilled in the art, a single bipolar electrode may be substituted for the pair of electrodes 118. In applications where cooling of the fluid is required, a cooling rod or other cryogenic element using conventional cooling methods may be substituted for the heating elements. As the conductive fluid (e.g., saline) completes the circuit between the electrodes 118, the current flowing therebetween heats the fluid to a desired temperature (e.g., approximately 90° C.). A prime port 115 for initializing fluid into the system 100 is formed at lower end of the heating column 114 and the heating column 114 is fluidly coupled to an inlet 117 of the pump 116. Fluid passes through the pump 116 to an outlet 119 which is fluidly coupled to a supply lumen of the probe body 102 to pass therethough into the uterus 110. For example, the probe body 102 may include an annular supply lumen surrounding by a central return lumen. Fluid is withdrawn from the uterus 110 into the return lumen of the probe body 102 to pass to a fluid return port 122 at an upper end of the heating column 114. The fluid returned from the uterus 110 via the return port 122 passes through the heating column 114 back to the inlet 117 of the pump 116 to return to the uterus 110. The fluid is circulated through this circuit with additional fluid from the bag 108 replacing any fluids lost (e.g., through absorption, etc.) until the procedure has been completed. Once the procedure is complete, the outlet port 109 is opened to permit the fluid to flow into the drainage bag 112.

In addition, the system 100 according to this embodiment includes an air venting port 160 which may be used to purge air from the system 100. The venting port 160 may include a hydrophobic filter 158 to prevent fluids from being vented therethrough.

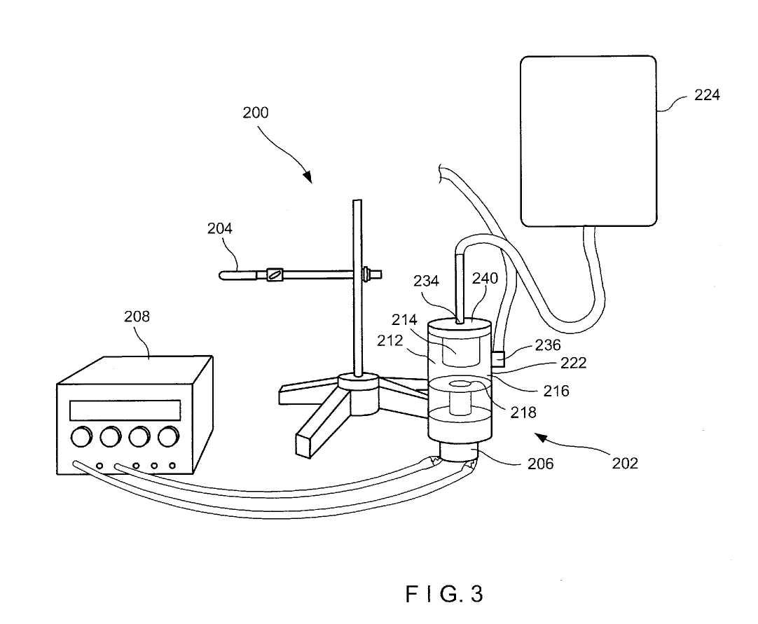

A hand-held thermal ablation device 200 according to a second embodiment of the invention is shown in FIG. 3. The hand-held thermal ablation device 200 utilizes a non-displacement centrifugal pump to circulate the fluid through the uterus. Using a centrifugal pump rather than a positive displacement pump allows the selection of a dedhead pressure lower than a threshold pressure which risks forcing the fallopian tubes open reducing the risk of damage to non-targeted tissue. In addition, a centrifugal pump may be less affected by debris in the flow (e.g., tissue debris) and less susceptible to over-pressurization of the outflow due to blockage in the pump.

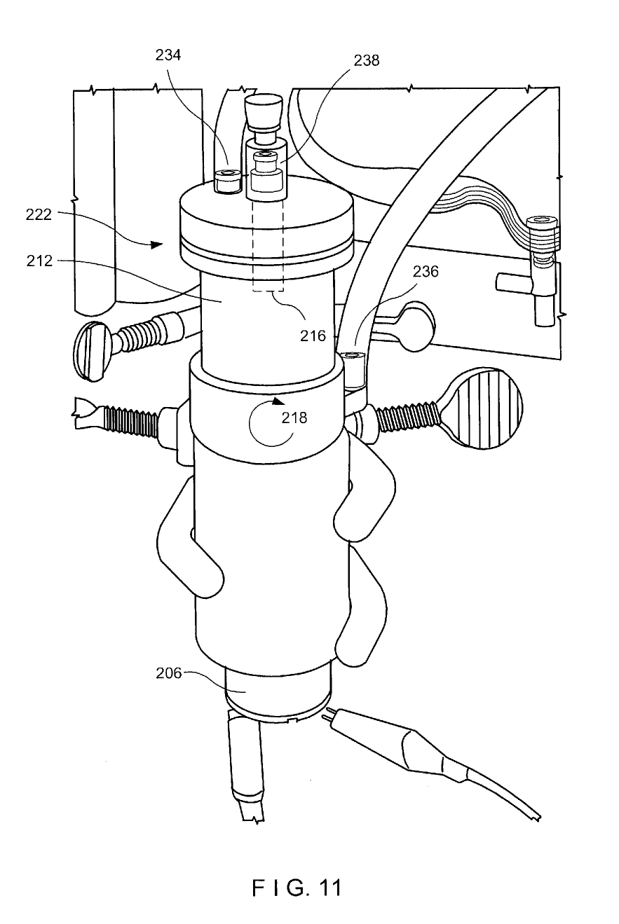

As with the previous embodiment, the device 200 includes an elongated probe 204 adapted for insertion through the cervix into the uterus extending from a handle 202. As would be understood by those skilled in the art, a fluid supply bag 224 may be connected to the inlet 234 via tubing, to provide a supply of fluid which fills a fluid reservoir 212 from which solid debris is filtered out. The handle 202 comprises a DC motor 206, which is preferably a brushless motor, electrically connected to a controller such as a DC power supply 208 for driving a pump 222. As would be understood by those skilled in the art, a temperature probe (e.g., an electronic temperature probe) may be provided in the flow path (e.g., within the pump 222) to monitor fluid temperature. In addition, a fluid reservoir 212 with a debris trap 214 may be incorporated into the pump housing 222 to remove particulate matter (e.g., tissue) to clean fluid returning to the pump 222 from the uterus. A heater column 216 is incorporated into the pump 222 as shown in more detail in FIG. 11.

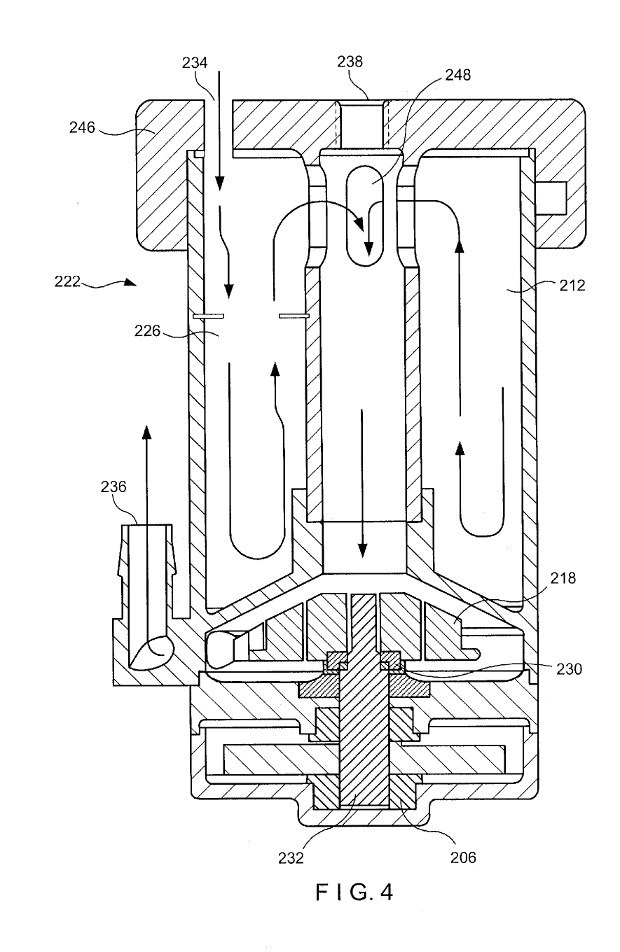

The pump housing 222 is shown in greater detail in FIGS. 4-12. The fluid column 226 extends generally through the center of the housing 222, and contains a heating element which in this embodiment is formed as a heating column 216. A centrifugal pump impeller 218 disposed at the lower part of the pump housing 222 is connected to the electric motor 206 via a coupling 232 which, in this embodiment, comprises a drive shaft with a seal assembly 230. Alternatively, as would be understood by those skilled in the art, a magnetic coupling may be used as the coupling 232 obviating the need for a seal as no shaft would need to pass through the walls of the housing 222 in this case.

As shown by the arrows in FIG. 4, fluid enters the pump housing 222 via a fluid inlet 234 formed in a top cover 240 of the housing 222. The fluid enters a top of the fluid column 226 via an inlet 248, is heated by the heating column 216 and enters the impeller 218 where it is accelerated, pressurized and discharged through a fluid outlet 236 connected to the elongated probe 204 via tubing.

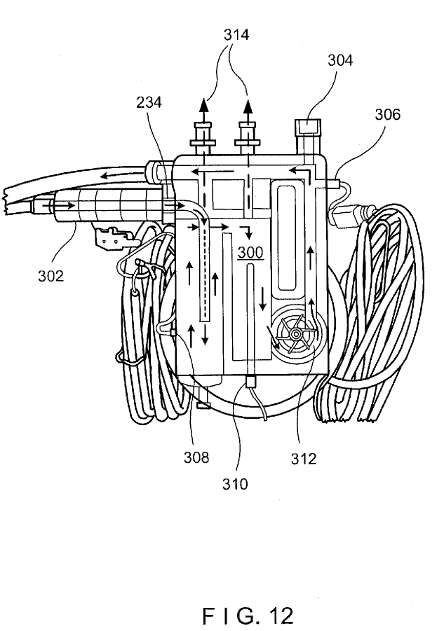

As shown in FIG. 12, an exemplary suite of sensors which may be used in a hand-held device according to the invention includes a flow sensor 302 disposed, for example, adjacent to the return port 234 of a pump 300 and a pressure sensor 304 may measuring the pressure of fluid leaving the pump 300 to the elongated probe. The temperature of the fluid may be measured at the exit from the pump by a temperature sensor 306, and at the return to the pump with a temperature sensor 308. Furthermore, the pump 300 may include air venting ports 314 which may be used to purge air from the pump 300. Additional electrical connections may be used to provide power to the device. For example, electrical leads 310 and 312 may be used to power respectively the heating elements and the pump of the device.

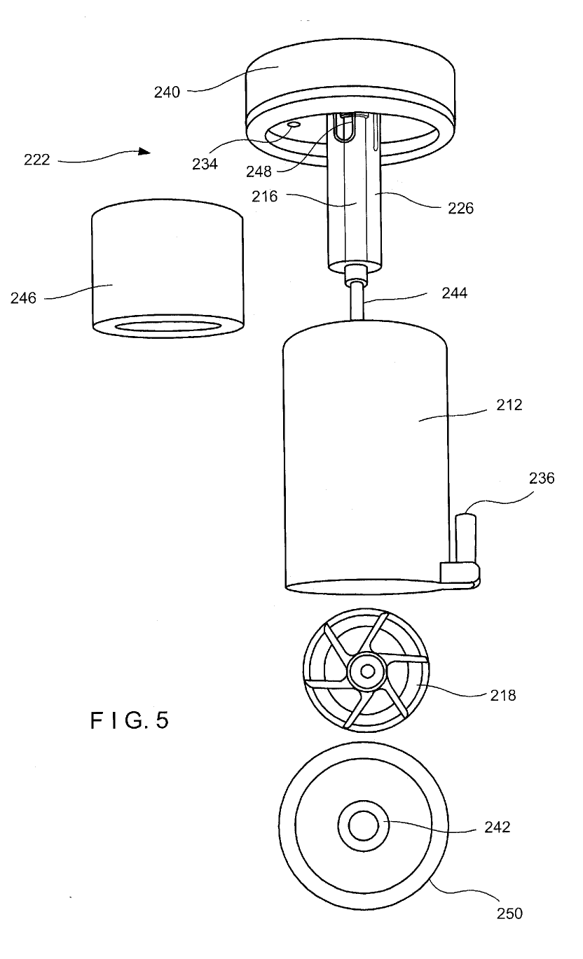

FIG. 5 shows an exploded view of the pump housing 222 with the reservoir 212. In an exemplary embodiment, the pump housing 222 may be constructed of high temperature polycarbonate or polysulfone. The cap or cover 240 includes the inlet port 234 and the inlet 248 to the fluid column 226. The exemplary bipolar RF electrode 244 forms the heating element of the heating column 216, and is disposed concentrically to the reservoir 212 that is designed to separate bubbles and debris from the fluid. Those skilled in the art will understand that this heating element is only one exemplary embodiment and that any suitable mechanism for heating the fluid may be included in the devices according to the invention. A macro filter 246 is provided to remove from the liquid pieces of biological tissue and blood clots that may be aspired by the pump.

The fluid is motivated by the impeller 218 that is mounted on a lower housing cap 250 with a bearing 242 and sealing elements. A shaft may pass through the opening of the bearing 242, however the lower housing may be sealed in a different embodiment using a magnetic coupling. A fluid outflow port 236 is located in the high pressure side of the pump, to provide pressurized fluid to the elongated probe 204 and to the patient.

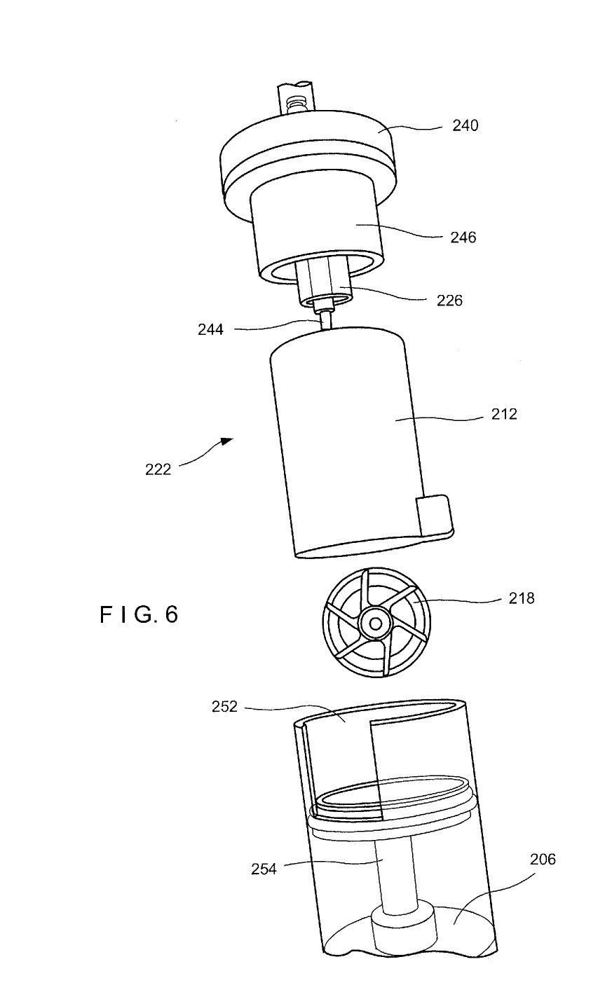

The reservoir 212 and the motor housing 252 are shown in greater detail in FIG. 6. The motor housing 252 interfaces with the fluid reservoir 212 at the top, and with the exemplary brushless DC motor 206 at the bottom. The impeller shaft 254 extends through the bearing 242 of the lower cover 250, such that in this exemplary embodiment the impeller 218 is mechanically coupled to the motor 206. Alternatively, a drive shaft of the motor may be directly coupled to the impeller with appropriate seals therearound as would be understood by those skilled in the art. A magnetic coupling may be used in a different embodiment, for example comprising a magnet or magnetic disk on or about the impeller 218 and opposite coupling means on or about the motor 206.

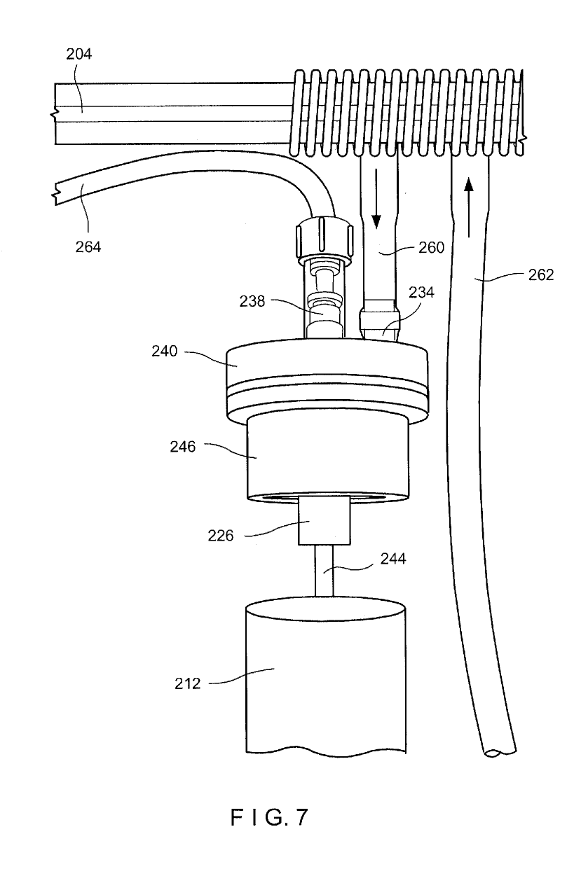

FIG. 7 shows a close up of the fluid reservoir system according to an embodiment of the invention. The exemplary bipolar electrode 244 is electrically coupled to an RF power supply via the RF power conductor 264. The fluid entering the fluid reservoir 212 passes through the macro filter 246, as described above, that is disposed on the outside of the fluid column 226. Fluid thus fills the reservoir 212 until it overflows and starts spilling into the center column 226 through inlet drain holes 248. Within the center column 226, the fluid is directed along the RF electrode 244, where it is heated.

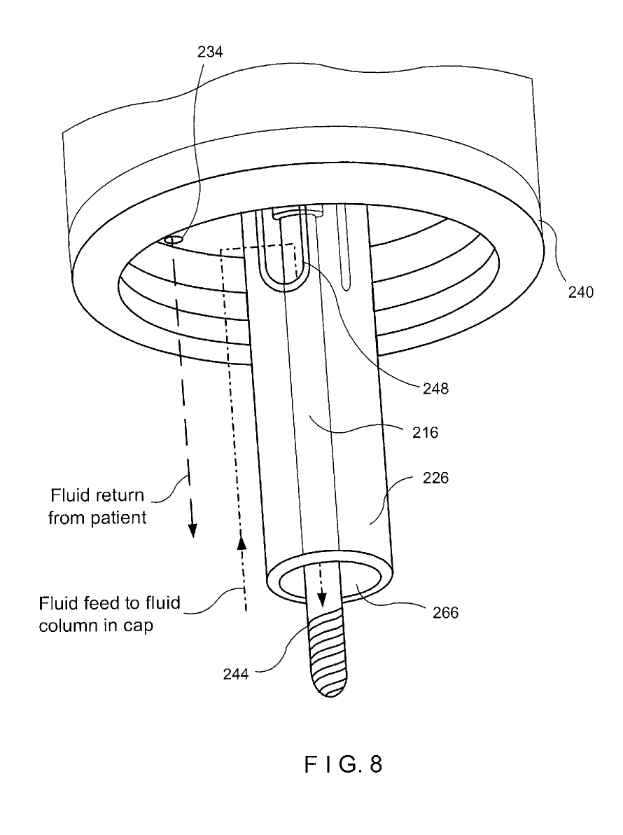

As the fluid passes along the center column 226, the ions in the saline (or other suitable fluid) carry the current and are excited, thus heating the fluid. FIG. 8 shows a detailed view of the fluid heating column 216 integrated into the cap 240 and the fluid column 226. The fluid path directs the heated fluid through the centrifugal pump inlet 266 into the low pressure side of the impeller 218.

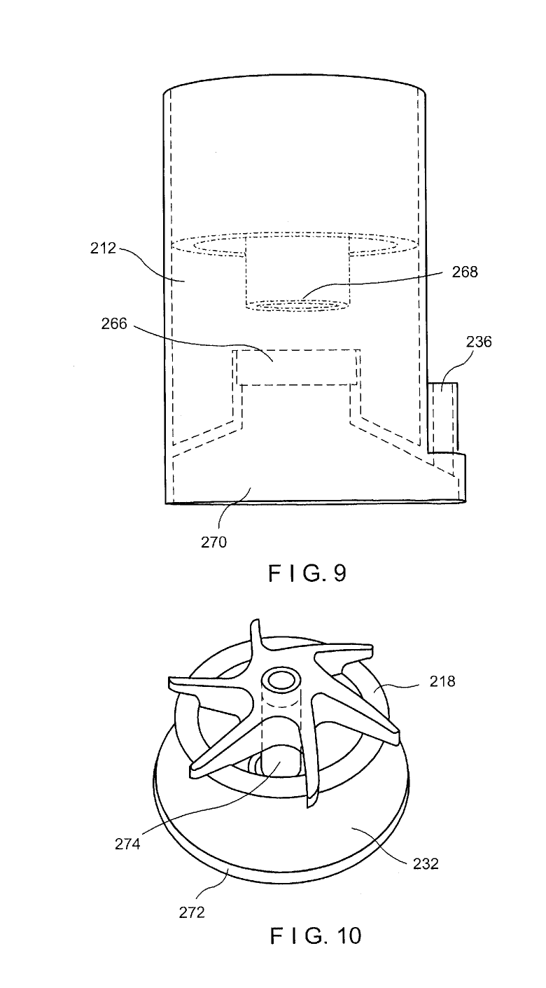

FIG. 9 shows a detailed diagram of the reservoir 212 and its components. The relationship between the pump inlet 266, pump outlet 236 and fluid column attachment point 268 can be seen within the reservoir 212. In addition, the reservoir may include an impeller section 270 for housing the impeller 218. FIG. 10 shows a detailed view of the centrifugal pump impeller. The impeller 218 is connected to the coupling 232, that in this exemplary case may be a magnetic coupling. The shaft 274 connects the impeller 218 to a magnetic disk 272, which is magnetically coupled to a similar apparatus attached to the motor.

In a different embodiment, the hand-held thermal ablation device of the invention may use a single pass flow path rather than recirculating the fluid from the uterus of the patient. For example, the saline bag used to start the system in the embodiment described above may be fluidly connected to the heater column that is in turn connected to the pump. The fluid from the pump is then routed to the uterus where it performs the therapeutic function. Instead of returning to the device, fluid from the uterus is discharged into a collection bag for disposal. Without fluid recirculation, the filter and debris catch described above are not necessary. The fluid reservoir may also be smaller or completely removed.

According to embodiments of the invention, the fluid circulated by the hand-held thermal ablation device may contain therapeutic compounds as necessary. For example, drugs and medications may be added to the ablation fluid or may be circulated separately from the ablation fluid. The saline, glycerin or other fluid used for the thermal therapy may be used as a carrier for the drugs during the ablation procedure, or alternatively may be used without heating to transport the drugs. As would be understood by those skilled in the art, for applications requiring heated fluid and utilizing RF energy for the heating, the fluid used must be electrically conductive.

Those of skill in the art will understand that the thermal ablation system according to the invention is not limited to use within the uterus. Other hollow organs and structures within the body may be treated by liquid hyperthermia and/or hypothermia. For example, the bladder, kidneys, intestines etc. can be flushed with circulating hot or cold fluids provided by the hand-held device according to the invention. In particular, a heated fluid may improve the absorption of medications contained therein by the walls of the vessel being treated, increasing the therapeutic benefit.

Application of a heated fluid to a target tissue may be used to destroy the lining of the vessel, for example to stop bleeding, or to control the absorption of drugs by the tissue. Hypothermia treatment using a cooling rod in the device may be beneficial for the control of bleeding, to reduce blood flow to target tissue, or for temperature controlled drug activation, for example.

When the hand-held thermal ablation system according to the invention is used for certain tubular organs such as the intestine, leakage from the organ may be a problem. For example, devices to occlude the organ and prevent the fluid from escaping may be incorporated in the elongated probe introduced into the organ. In one embodiment, a pair of occluding compliant balloons may be used to close off the portions of the organ being treated.

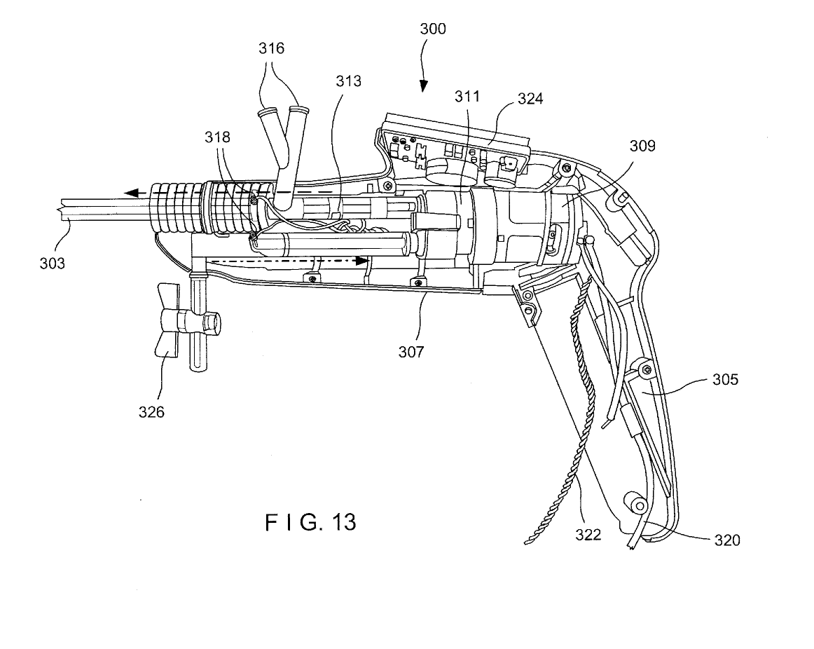

FIG. 13 shows an exemplary embodiment of the components of the heat treatment device according to the invention, integrated into a handle usable during surgical procedures. The exemplary hand-held thermal ablation device 300 comprises a housing 307 connected to a fluid sheath or elongated probe 303 adapted for insertion into the patient. The housing 307 has a handle portion 305 that the physician can grasp to maneuver and operate the device. An electronic module 324 may be provided, containing a display for the pressure, temperature and any other desired parameters, as well as electrical circuits to control the device.

An electric motor 309 is disposed within the housing 307, and is coupled to an impeller 311. In this exemplary embodiment, the preferred pump 311 is a centrifugal pump. However a displacement pump may also be used in the device if controls are incorporated preventing the pump from over-pressurizing the uterus. After exiting the pump 311 the fluid is heated by a heating element 313. As described above, the heating element 313 may comprise monopole or dipole electrodes or other heating devices. The fluid enters the device 300 via prime ports 316, and after heating circulates to the patient via a fluid sheath 303. Furthermore, as would be understood by those skilled in the art, additional fluid may be added as needed via the prime ports 316 to compensate for uterine distension and any fluid absorption in the uterus. An RF cable 320 provides RF power supply to the heating element 312 while a DC motor power cable 322 provides DC current to the pump 310. The system has a temperature sensing system 318 including two temperature sensors—“thermistors” that monitor fluid temperature. As seen in FIG. 13, the temperature sensing system 318 includes a top sensor measuring the temperature of fluid flowing out to the patient while the bottom sensor measures the temperature of fluid returning from the patient to the device. In addition, a drain unit 326 is coupled to the fluid sheath 303 to bleed fluid therefrom if desired.

The present invention was described with reference to specific exemplary embodiments. Those skilled in the art will understand that changes may be made in details, particularly in matters of shape, size, material and arrangement of parts. For example, the invention is not limited to methods and devices for the thermal ablation of the uterine lining. Accordingly, various modifications and changes may be made to the embodiments. The specifications and drawings are, therefore, to be regarded in an illustrative rather than a restrictive sense.

What is claimed is:

1. A thermal ablation system, comprising:
a device housing;
an elongated probe extending distally from the device housing, the probe including an outflow fluid passage extending between proximal and distal outflow openings and a return fluid passage extending between proximal and distal return openings, the probe being shaped and sized for insertion into a body lumen so that, when the distal outflow and return openings are located at a desired position within the body, the proximal outflow and return openings remain outside the body;
a pump disposed in the device housing in fluid communication with the outflow and return fluid passages of the probe for circulating a fluid through the outflow lumen into a target area of the body and back through the return lumen to the device housing;
a heating element in the device housing for heating the fluid; and
fluid connectors placing the pump and the heating element in fluid communication with a supply of fluid and a fluid drain.

2. The thermal ablation system according to claim 1, further comprising a pump housing disposed in the device housing, the pump housing comprising a fluid reservoir, a debris trap and a filter.

3. The thermal ablation system according to claim 1, wherein the pump is a centrifugal pump.

4. The thermal ablation system according to claim 1, wherein the pump is an oscillating pump.

5. The thermal ablation system according to claim 2, wherein the pump housing comprises an impeller of a centrifugal pump.

6. The thermal ablation system according to claim 2, further comprising a magnetic coupling between a pump motor in the device housing and an impeller in the pump housing.

7. The thermal ablation system according to claim 2, further comprising a heater column disposed in the pump housing, the heater column containing fluid flowing over the heating element.

8. The thermal ablation system according to claim 1, wherein the heating element comprises one of monopolar and bipolar RF electrodes.

9. The thermal ablation system according to claim 1, wherein the outflow and return fluid passages of the probe are substantially coaxial and wherein the distal outflow and return openings are substantially coaxial.

10. The thermal ablation system according to claim 1, further comprising pressure and temperature feedback loops maintaining a pressure and temperature of the fluid below predetermined threshold levels.

11. The thermal ablation system according to claim 1, wherein the elongated probe comprises sealing elements which, when the distal outflow and return openings are in a desired position within a uterus, seal against a cervical os.

12. A hand-held thermal ablation device, comprising:
a handle;
an elongated probe extending between a proximal end coupled to the handle and a distal end which, when in an operative position, is received within a body lumen;
a pump within the handle in fluid connection with fluid passages in the elongated probe;
a fluid column within the handle containing a heating element, serially connected with the pump; and
an external fluid supply in fluid connection with the pump.

13. The hand-held thermal ablation device according to claim 12, wherein the probe is sized and shaped for insertion through a cervix into a uterus.

14. The hand-held thermal ablation device according to claim 12, wherein the pump housing in the handle, the pump housing comprising a fluid reservoir, the fluid column and a filtering element.

15. The hand-held thermal ablation device according to claim 14, further comprising an impeller disposed in the pump housing magnetically coupled to a motor.

16. The hand-held thermal ablation device according to claim 12, wherein the heating element is selected to heat the fluid to between 41.5° C. and 99.9° C.

17. The hand-held thermal ablation device according to claim 16, wherein the heating element is selected to heat the fluid to about 90° C.

18. The hand-held thermal ablation device according to claim 14, further comprising a drainage passage receiving fluid from the return passage for discharge into a disposal container.

19. A method for ablating target tissue, comprising:
advancing a distal end of an elongated probe of a hand-held device into a body lumen;
heating a fluid with a heating element disposed in a housing of the hand-held device;
motivating the fluid with a pump disposed in the housing, to inject the fluid into the body lumen via an outflow passage of the elongated probe to ablate target tissue therein; and
withdrawing the fluid from the body lumen via a return passage of the elongated probe.

20. The method according to claim 19, further comprising advancing the distal end of the elongated probe through a cervix into a uterus.

21. The method according to claim 19, further comprising heating the fluid to a temperature of between about 41.5° C. and about 99.9° C.

22. The method according to claim 19, further comprising heating the fluid to a temperature of about 90° C. for approximately 10 minutes.

23. The method according to claim 22, further comprising monitoring the pressure and temperature in a feedback loop to stop the pump if selected parameters are exceeded.
